(19) United States
(12) Patent Application Publication
(10) Pub. No.: US 2024/0350307 A1
(43) Pub. Date: Oct. 24, 2024

(54) Retina Clip

(71) Applicant: The Board of Trustees of the Leland Stanford Junior University, Stanford, CA (US)

(72) Inventors: Eduardo Jose Chichilnisky, Stanford, CA (US); Ruwan Amila Silva, Palo Alto, CA (US); Martin Breidenbach, Los Altos, CA (US); Knut Skarpaas, VIII, Menlo Park, CA (US); Marco Oriunno, Menlo Park, CA (US)

(73) Assignee: The Board of Trustees of the Leland Stanford Junior University, Stanford, CA (US)

(21) Appl. No.: 18/642,416

(22) Filed: Apr. 22, 2024

Related U.S. Application Data
(60) Provisional application No. 63/497,673, filed on Apr. 21, 2023.

(51) Int. Cl. A61F 9/007

(52) U.S. Cl. CPC A61F 9/00727; A61F 2220/0033

(57) ABSTRACT

A retina clip in accordance with embodiments of the invention are illustrated. One embodiment includes a retina clip including an outer ring having a means of retention against the retina, and an inner platform configured to hold a payload against the retina of an eye, where the inner platform is suspended from the inner ring via several springs. In many embodiments, the springs are flexures. The springs can be modified to generate a desired deflection based on a given payload attached to the inner platform. In numerous embodiments, the payload is a microelectrode array used as part of a retinal prosthetic.

### CROSS-REFERENCE TO RELATED APPLICATIONS

The current application claims the benefit of and priority under 35 U.S.C. § 119(e) to U.S. Provisional Patent Application No. 63/497,673 entitled “Retina Clip” filed Apr. 21, 2023. The disclosure of U.S. Provisional Patent Application No. 63/497,673 is hereby incorporated by reference in its entirety for all purposes.

### GOVERNMENT LICENSE RIGHTS

This invention was made with Government support under contract EY032900 awarded by the National Institutes of Health. The Government has certain rights in the invention.

### FIELD OF THE INVENTION

The present invention generally relates to retina clips, namely implantable structures for securing retinal prosthetic electronics inside the eye.

### BACKGROUND

The human eye is the organ responsible for sight. The eye is an optical device made up of several layers. The innermost, light-sensitive layer of the eye is the retina. The retina consists of several layers of photoreceptors and neurons that receive light and transmit visual information to the brain. Damage to the retina can result in loss or reduction in vision. Two common eye diseases, retinitis pigmentosa and age-related macular degeneration, cause many cells in the retina to die. Retinal prosthetics are a class of medical devices designed to interface with the neurons in the retina in order to restore vision.

### SUMMARY OF THE INVENTION

A retina clip in accordance with embodiments of the invention are illustrated. One embodiment includes a retina clip including an outer ring having several retention members, and an inner platform configured to hold a payload against a retina of an eye, where the inner platform is suspended from the inner ring via several springs.

In a further embodiment, the outer ring includes several eyelets, and the retention members of the several retention members are spikes that fit through the several eyelets.

In still another embodiment, the several eyelets contain three eyelets spaced 120 degrees apart on an outer edge of the outer ring.

In a still further embodiment, the retention members of the several retention members are spikes.

In yet another embodiment, the several retention members include 6 spikes spaced 60 degrees apart on an outer edge of the outer ring.

In a yet further embodiment, the several springs contain three springs.

In another additional embodiment, the outer ring includes three eyelets spaced 120 degrees apart on an outer edge of the outer ring, and wherein each spring of the three springs is attached equally spaced between two of the three eyelets.

In a further additional embodiment, the outer ring, the inner platform, and the several springs are made of nitinol.

In another embodiment again, lengths of the springs in the several springs point towards the inner platform.

In a further embodiment again, lengths of the springs in the several springs point are orthogonal to the inner platform.

In still yet another embodiment, the payload is a microelectrode array.

In a still yet further embodiment, the payload is a microLED array.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A illustrates a perspective view of a first retina clip in accordance with an embodiment of the invention.

FIG. 1B illustrates a top view of the first retina clip in accordance with an embodiment of the invention.

FIG. 1C illustrates a side view of the first retina clip in accordance with an embodiment of the invention.

FIG. 1D illustrates a bottom view of the first retina clip in accordance with an embodiment of the invention.

FIG. 2A illustrates a perspective view of a second retina clip in accordance with an embodiment of the invention.

FIG. 2B illustrates a top view of the second retina clip in accordance with an embodiment of the invention.

FIG. 2C illustrates a side view of the second retina clip in accordance with an embodiment of the invention.

FIG. 2D illustrates a bottom view of the second retina clip in accordance with an embodiment of the invention.

FIG. 3A illustrates a perspective view of a third retina clip in accordance with an embodiment of the invention.

FIG. 3B illustrates a top view of the second retina clip in accordance with an embodiment of the invention.

FIG. 3C illustrates a side view of the second retina clip in accordance with an embodiment of the invention.

### DETAILED DESCRIPTION

Retinal prosthetics are devices that use electrodes, microLEDs, or other neurostimulation methods to stimulate retinal cells in a specific manner to evoke visual perception of the surrounding environment in patients who have lost their natural sight. One means of stimulating retinal cells occurs via an array of electrodes that are implanted into the eye against the retina. Typically, the electrode arrays have a cable leading away towards a controller. In order to keep the array in place, a tack is placed through the cable near the electrode array. This tack is relied upon to retain the array against the retina. This retention method has significant disadvantages.

Due to the single point of retention, the electrode array is not maintained in a fixed position with respect to the retina in any dimension. Not only can the array drift across the retina, it can also drift away from the retina. This change in position can significantly reduce efficacy of the prosthetic as specific electrodes in the array are meant to stably stimulate specific retinal cells. When the array moves, it is no longer known which cells are adjacent to which electrode in the array, and therefore the entire device must be recalibrated.

Further, the aforementioned drift can change the pressure with which the array presses against the retina reducing (sometimes completely) the ability of these electrodes to stimulate retinal cells. Additionally, the tack being at only one end of the array can cause a pressure gradient with respect to the retina across the array. In order to resolve these issues, a clip for attaching prosthetic components to the retina is described. In many embodiments, the clip is circular having an outer ring and an inner platform. The outer ring has retention members around the outer ring. In numerous embodiments, the outer ring is fabricated with spikes that are designed to be placed through the eye to retain the ring. In various embodiments, eyelets around the outer ring through which retinal tacks can be placed in order to hold the clip to the retina. In many embodiments, three eyelets and three corresponding tacks are used. Fewer tacks can be used, but retention in one of the three dimensions may be impacted. Similarly, more tacks can be used depending on the needs of a particular patient. Additionally, other retention methods that do not involve spikes or tacks can be used without departing from the scope or spirit of the invention.

The inner platform is connected to the outer ring via springs. The inner platform is configured to hold a payload such as (but not limited to) an electrode array. In many embodiments, the spring coefficient for the springs can be modified based on the particular payload in order to control the displacement of the payload. In numerous embodiments, the number of turns in the spring are modified. In various embodiments, the payload can be combined with a spacer in order to control the displacement of the payload. In a variety of embodiments, the springs are configured to exert a controlled pressure of approximately 1 mg/mm2, however the number of convolutions and/or other spring parameters can be modified to generate a pressure appropriate for a given payload.

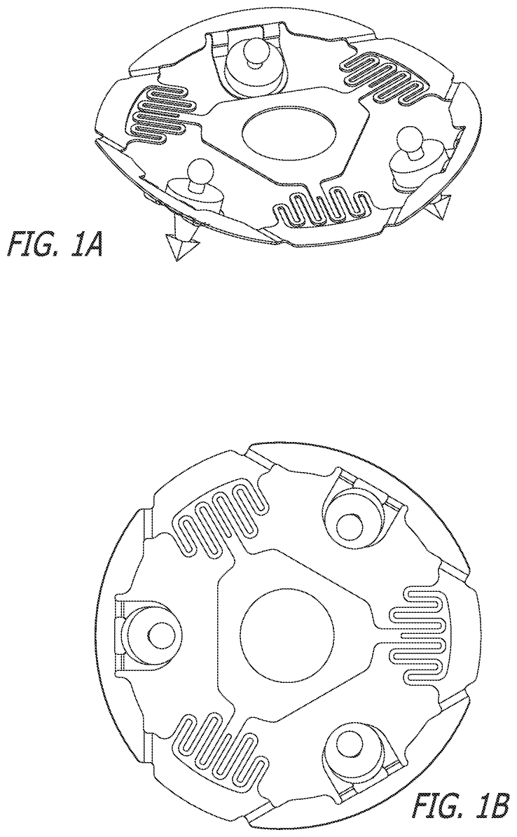

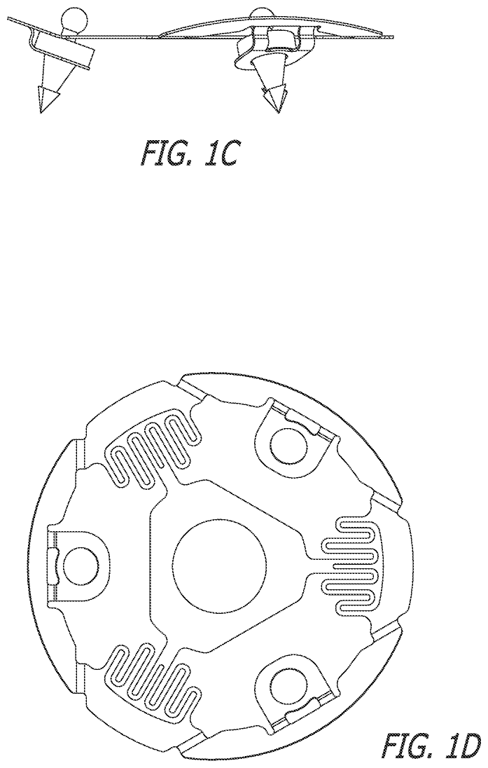

In various embodiments, the springs are designed such that the length of the spring points inwards towards the inner platform. In numerous embodiments, the springs are flexure springs. When force is applied to an object, the object deflects with the path of least resistance to absorb the associated stress. In flexures, there are usually designated areas that are made very narrow to assure that motion happens in a controlled way. Because many retinal clips are very thin, there is a limit to how narrow one can easily make a structural beam. For this thin part, it can be difficult to get an aspect ratio for the ligament which will keep the serpentine flexure spring in a plane. As extra convolutions are added to the serpentine to reduce the spring constant, the out of plane motion becomes increasingly unpredictable. When all deflections must be predictable, the orientation of the spring can be flipped so that the length of the spring is orthogonal to the edge of the inner platform (as seen in FIG. 1A-D). This spring orientation forces the inner platform out of plane in a predictable manner by using the bending of symmetric cantilever sections along with a little rotation by torsion to achieve the required motion. This can also make the part insensitive to manufacturing limitations.

In many embodiments, the clip can be folded for insertion into the eye and unfurled once placed inside the eye. In numerous embodiments, the retina clip is made of nitinol. However, any material with sufficient biocompatibility may be used in a similar fashion. In various embodiments, the retina clip is curved such that that outer ring is not pressed against the retina while the eyelets are. While the clip can be made smaller or bigger as appropriate to the requirements of specific applications of embodiments of the invention. In many embodiments, the clip is approximately 8 mm in diameter. However, the size may be modified depending on the payload to be delivered, and the size of the eye.

Turning now to the figures, FIGS. 1A-1D, a retinal clip in accordance with an embodiment of the invention is illustrated. Clip 100 includes 3 eyelets 110 offset by 120 degrees around the outer ring 120. Springs 130 are orthogonal to, and suspend, the inner platform 140.

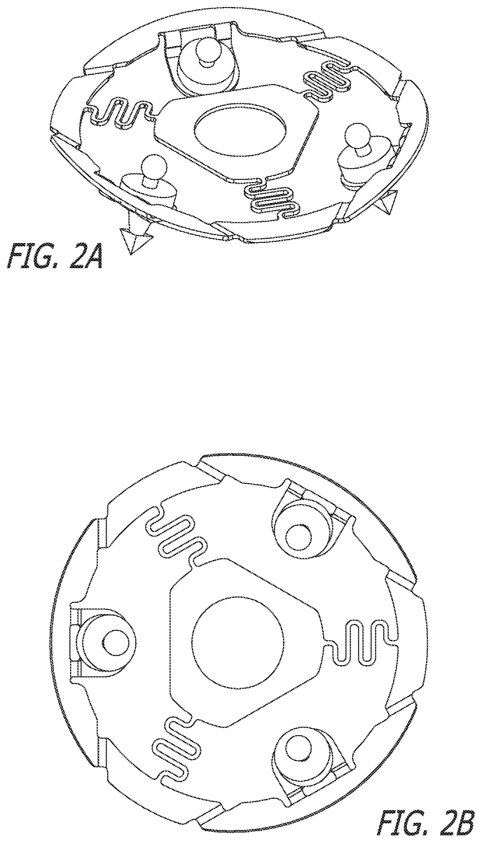

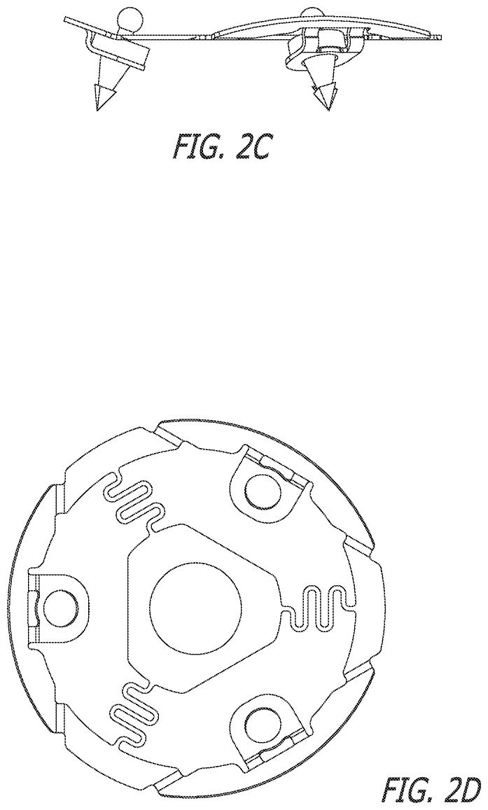

FIGS. 2A-2D illustrate another retinal clip in accordance with an embodiment of the invention. Clip 200 includes 3 eyelets 210 offset by 120 degrees around the outer ring 220. Springs 230 point towards, and suspend, the inner platform 240.

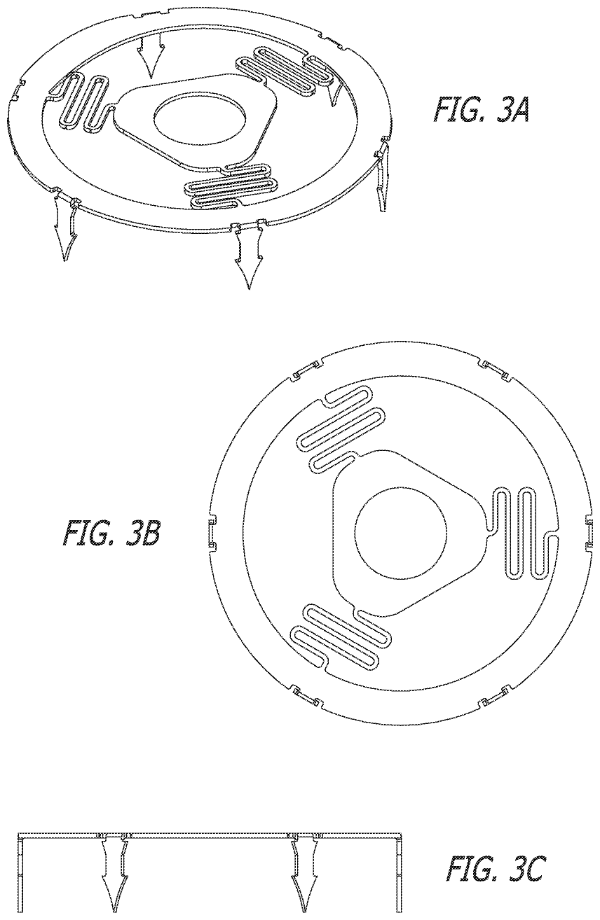

FIGS. 3A-3C illustrate yet another retinal clip in accordance with an embodiment of the invention. Clip 300 includes 6 spikes 310 equally offset around the outer ring 320. Springs 330 point towards, and suspend, the inner platform 340.

While three particular embodiments are illustrated in FIGS. 1A-3C, it is readily appreciated that certain features may be modified to produce additional embodiments. For example, a clip may have spikes yet also have springs that are orthogonal to, and suspend, the inner platform. Similarly, different numbers of eyelets or spikes may be used. Further, different numbers of convolutions in the springs (or a different number of springs) compared to those illustrated may be used to generate the appropriate force for a given payload.

Indeed, although specific retinal clips are discussed above, many different clip designs can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A retina clip comprising:
an outer ring having a plurality of retention members; and
an inner platform configured to hold a payload against a retina of an eye;
where the inner platform is suspended from the inner ring via a plurality of springs.

2. The retina clip of claim 1, wherein the outer ring comprises a plurality of eyelets, and the retention members of the plurality of retention members are spikes that fit through the plurality of eyelets.

3. The retina clip of claim 2, wherein the plurality of eyelets contains three eyelets spaced 120 degrees apart on an outer edge of the outer ring.

4. The retina clip of claim 1, wherein the retention members of the plurality of retention members are spikes.

5. The retina clip of claim 4, wherein the plurality of retention members comprises 6 spikes spaced 60 degrees apart on an outer edge of the outer ring.

6. The retina clip of claim 1, wherein the plurality of springs contains three springs.

7. The retina clip of claim 1, wherein the outer ring comprises three eyelets spaced 120 degrees apart on an outer edge of the outer ring, and wherein each spring of the three springs is attached equally spaced between two of the three eyelets.

8. The retina clip of claim 1, wherein the outer ring, the inner platform, and the plurality of springs are made of nitinol.

9. The retina clip of claim 1, wherein lengths of the springs in the plurality of springs point towards the inner platform.

10. The retina clip of claim 1, wherein lengths of the springs in the plurality of springs point are orthogonal to the inner platform.

11. The retina clip of claim 1, wherein the payload is a microelectrode array.

12. The retina clip of claim 1, wherein the payload is a microLED array.
